(19) United States
(12) Patent Application Publication
(10) Pub. No.: US 2024/0402155 A1
(43) Pub. Date: Dec. 5, 2024

(54) Fluid-Encapsulated Eukaryotic-Cell Model

(71) Applicant: Junnis Hugger, III, Tallahassee, FL (US)

(72) Inventor: Junnis Hugger, III, Tallahassee, FL (US)

(21) Appl. No.: 18/732,513

(22) Filed: Jun. 3, 2024

Related U.S. Application Data
(60) Provisional application No. 63/505,983, filed on Jun. 2, 2023.

(51) Int. Cl. G01N 33/50

(52) U.S. Cl. CPC G01N 33/5076

(57) ABSTRACT

A fluid-encapsulated eukaryotic-cell model is a synthetic cell within a liquid environment that includes a semi-permeable membrane and a plurality of artificial organelles. The semi-permeable membrane is immersed within the liquid environment so that an encapsulated portion of liquid from the liquid environment becomes located within the semi-permeable membrane. The encapsulated portion of liquid and the plurality of artificial organelles are housed within the semi-permeable membrane, which allows the plurality of artificial organelles to be suspended within the semi-permeable membrane by the encapsulated portion of liquid.

The current application claims a priority to the U.S. provisional patent application Ser. No. 63/505,983 filed on Jun. 2, 2023. The current application is filed on Jun. 3, 2024, while Jun. 2, 2024 was on a weekend.

### FIELD OF THE INVENTION

The present invention generally relates to biotechnology under synthetic biology. More specifically, the present invention relates to a fluid-encapsulated cell model that incorporates the presence of 3D-printed organelles.

### BACKGROUND OF THE INVENTION

The present invention applies to the understood practices in synthetic biology, molecular biology, biotechnology, and education. In the development of this model and in biology, a cell is the fundamental unit of life that constitutes all living beings. In Synthetic biology and biology courses the structure and function of a cell stands as a rudimentary introduction to the foundation of a studying life. As the student progresses through their career the same conceptual framework comes into discussion a bountiful number of times. In the classroom, this leaves teachers seeking methods to cram large portions of material into expressive lessons to emphasize the core areas that the class will focus on. This leads those educators, focused on retention, engagement, and advanced material to rely on modeling and experimentation to advance their effort. Currently, the two methods of modeling are live or static. In live models, educators employ this practice under microscopic viewing of model organisms. In doing so, these model organisms act as visual aids for cellular processes and lack the ability to observe intramolecular interactions. As a luxury of well supplied education, students are able to view these models only under the conditions that the school or educator allocates the large amount of funding needed to hold live animals, the school is equipped in case of a biohazard, and the beliefs of the student do not conflict with the moral principles applied while working with live models. For these reasons, in addition to the inherent brevity, educators turn to modeling. Yet these models offer something static, bulky, and do not exist for experimental engagement. As educators across the world introduce students to the molecular machinery that drives cell activity, static modeling serves as a placeholder of biological parts that removes the educator's ability to discuss molecular changes while actively observing cellular behavior.

By looking at prior art multiple advancements have been seen in similar regards. For instance, a U.S. patent application 2007/0292830 A1 discloses collection of models for facilitating the study of at least one of a biological subject, an anatomical organ and an anatomical organ system of a living being. The collection of 41 three-dimensional products, 11 raised products, and a process of didactic interpretation in printed form and in Braille aim at the educational inclusion and improvement of the teaching-learning process of Morphological Sciences, in general, and especially for the visually handicapped, facilitating the association of theoretical concepts to the perception of the form, dimension, topography and proportions of the organic structures being studied.

A U.S. patent application 2007/0243511 A1 relates to tactile cell model comprising: a plurality of organelle models configured to be manipulated by hand and arranged on a generally flat surface; where the plurality of organelle models is further configured to be arranged with respect to each other so as to represent the arrangement of a plurality of organelles of an actual biological cell. A tactile biological cell model kit comprising: a cell membrane model; a cell wall model; a central vacuole membrane model; a nucleus model; a plurality of chloroplast models; a plurality of amyloplast models; a plurality of chromoplast models; a plurality of coccus bacteria models; a plurality of bacillus bacteria models; and where each of the models are configured to be manipulated by hand and arranged on a generally flat surface with respect to each other so as to represent the arrangement of a plurality of organelles of an actual biological cell. A tactile biological cell model kit comprising: a plurality of organelle models; and where each of the organelle models are configured to be manipulated by hand and arranged on a generally flat surface with respect to each other so as to represent the arrangement of a plurality of organelles of an actual biological cell.

A U.S. Pat. No. 8,460,005 B1 relates to kit for building a model of a cell. The method of creating a model of a biological cell. The method features providing a circular base with indentations disposed therein, forming a cell membrane and at least one organelle from a mixture formed by mixing all-purpose flour and water. Organelles may include a nuclear membrane, a cell wall, mitochondria, a smooth endoplasmic reticulum (ER), a rough ER, a vacuole, a Golgi apparatus, a nucleolus, a nucleoplasm, chromatin, a ribosome, DNA, RNA, transcription machinery, a histone, and a microtubule. The outside edge of the base is lined with a cell membrane. The organelles are inserted into indentations or atop the base. The organelles can be secured with glue.

A U.S. Pat. No. 4,312,826 relates to method for fabrication of physiological models. This invention relates to the method of producing a flexible, life-like model of physiological organs. These models are produced by injecting under pressure for a predetermined time a settable material such as silicone. The organ is then digested in a solution leaving a solid fabricated model. A multi-layered mold is fabricated around the solid model and once formed, is disassembled to remove said model. The mold is then reassembled and after proper coating is filled with liquid wax or similar material which is permitted to solidify. The mold is again disassembled, and the wax model removed. The surface of the wax model can then be polished and otherwise cleaned prior to dipping in a settable liquid such as silicone which allows a build-up coating to be applied to such wax model. The thus coated model is then placed in boiling water or other environment to melt out the core to form a hollow casting by the lost wax technique. The remaining waxy residue can be removed by use of an appropriate organic scavenger solution. In the case of lungs, heart and the like, the model can then be suspended within a suitable enclosure and through the application of an electronic controlled, alternating vacuum, respiration, heartbeat or the like can be simulated.

A U.S. Pat. No. 5,104,328 relates to anatomical model which represent the female abdominal area and include components simulating the layers of tissue in the abdominal area, the female reproductive organs, and the bladder. A simulated baby, umbilical cord, and placenta are also preferably included; and the tissue layer-representing components are provided with closures so that the dissecting and subsequent repairs of the tissue layers in surgical procedures can be simulated. The models are used to train professionals involved in surgical and to educate and inform persons, prospective parents, and others (i.e., both professionals and non-professionals) (e.g., with respect to such procedures).

The industry is continuously looking to generate ways for better learning, experimentation and well-structured models. The present invention aims to provide a fluid-encapsulated cell model that incorporates the presence of 3D-printed organelles. The organelles are designed to be housed in a spherical cell that allows the diffusion of small ions. The model is a transparent display of these organelles that is created in the process of spherification.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY OF THE INVENTION

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

An objective of the present invention is to provide an improved structure of encapsulated 3D-modeled organelles for educational and bio-technological purposes. More specifically, the principal objective of the present invention is to provide an artificial cell model that encapsulates unique 3D modeled organelles which designed to be housed in fluid environment for experimentation including that of the diffusion of small ions.

Another objective of the present invention is to provide a model with a transparent display for visual aid into the behavior of naturally occurring cells.

Another objective of the present invention is to provide a new synthetic cell which includes the housing of the 3D organelles with a liquid environment to allow the experimentation of complex molecular subjects.

Another objective of the present invention is to provide a model wherein the housing of the 3D organelles is in a liquid environment to allow the experimentation of complex molecular subjects

Another objective of the present invention is to provide a model that is flexible and suitable for vigorous experimentation

Other aspects, advantages and novel features of the present invention will become apparent from the detailed description of the invention when considered in conjunction with the accompanying drawings.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention and explain various principles and advantages of those embodiments.

FIG. 1 is a schematic view of the present invention.

FIG. 2 shows the first view of artificial biological cell with original 3D printed parts incorporating the embodiments of the invention.

FIG. 3 shows the second view of artificial biological cell with original 3D printed parts incorporating the embodiments of the invention.

FIG. 4 shows the third view of artificial biological cell with original 3D printed parts incorporating the embodiments of the invention.

FIG. 5 shows the fourth view of artificial biological cell with original 3D printed parts incorporating the embodiments of the invention.

FIG. 6 shows the fifth view of artificial biological cell with original 3D printed parts incorporating the embodiments of the invention.

FIG. 7 shows the sixth view of artificial biological cell with original 3D printed parts incorporating the embodiments of the invention.

The apparatus and method components have been represented where appropriate by conventional symbols in the drawings, showing only those specific details.

### DETAILED DESCRIPTION OF THE INVENTION

All illustrations of the drawings are for the purpose of describing selected versions of the present invention and are not intended to limit the scope of the present invention.

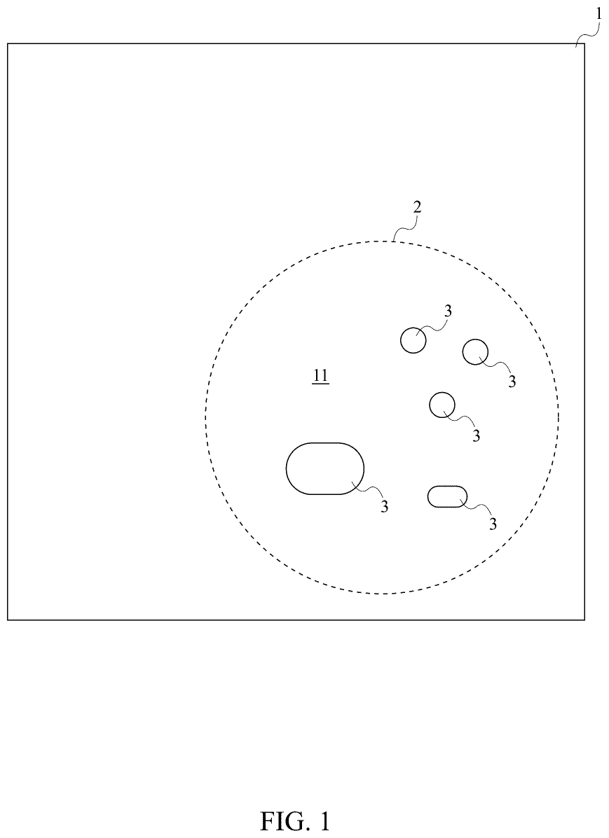

The present invention is a fluid-encapsulated eukaryotic-cell model that is used for educational and biotechnological purposes. As can be seen in FIG. 1, the present invention comprises a liquid environment 1, a semi-permeable membrane 2, and a plurality of artificial organelles 3. The liquid environment 1 allows the other components of the present invention to operate within a volume of liquid. The semi-permeable membrane 2 is an enclosure that only allows specific kinds of molecules to pass through itself. The plurality of artificial organelles 3 is a set of man-made objects that are meant to mimic organelles of a naturally-occurring eukaryotic cell. The plurality of artificial organelles 3 is preferably a plurality of three-dimensionally-printed (3D-printed) organelles.

The general configuration of the aforementioned components allows the present invention to mimic the biological processes of a naturally-occurring eukaryotic cell. The semi-permeable membrane 2 is immersed within the liquid environment 1, and an encapsulated portion of liquid 11 from the liquid environment 1 becomes located within the semi-permeable membrane 2, which allows the semi-permeable membrane 2 and its contents to operate through a liquid medium. The encapsulated portion of liquid 11 and the plurality of artificial organelles 3 are housed within the semi-permeable membrane 2 in order to complete a synthetic model of a eukaryotic cell. This also allows the plurality of artificial organelles 3 to be suspended within the semi-permeable membrane 2 by the encapsulated portion of liquid 11.

The semi-permeable membrane 2 can be modified through a variety of features in order to improve the functionality of the present invention. One feature is that the semi-permeable membrane 2 is preferably transparent so that its contents can be viewed from outside the semi-permeable membrane 2. Another feature is that the semi-permeable membrane 2 is preferably a spherical shape, which can be formed through a spherification process. Another feature is the semi-permeable membrane 2 is preferably made of a polymer (i.e., a polymeric semi-permeable membrane 2) and is more specifically made of an alginate polymer.

The semi-permeable membrane 2 is also preferably configured to bidirectionally diffuse small molecules (e.g., small ions) through a chemical mechanism used in cell encapsulation. Cell encapsulation is the process of enclosing a plurality of biological cells within a polymeric semi-permeable membrane 2, and the chemical mechanism used in cell encapsulation is a similar chemical mechanism used by the semi-permeable membrane 2 in order to bidirectionally diffuse small molecules. The chemical mechanism used in cell encapsulation is preferably a calcium-sodium reaction. Moreover, sodium alginate is dispersed throughout the liquid environment 1, and this sodium alginate is a medium for the chemical mechanism used in cell encapsulation.

The present invention may further comprise a depositing layer 4, which is preferably a generally-flat shape and would be used to further suspend the plurality of artificial organelles 3 within the semi-permeable membrane 2. The depositing layer 4 is mounted within the semi-permeable membrane 2, which allows the plurality of artificial organelles 3 to be mounted onto the depositing layer 4, so that the depositing layer 4 and the plurality of artificial organelles 3 are secured in place within the semi-permeable membrane 2. Moreover, the plurality of artificial organelles 3 is arranged with a specific interstitial spacing, and the specific interstitial spacing is proportional to an interstitial spacing amongst organelles of a naturally-occurring eukaryotic cell.

### Supplemental Description

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components but do not preclude the presence or addition of one or more other features, steps, operations, elements, components, and/or groups thereof.

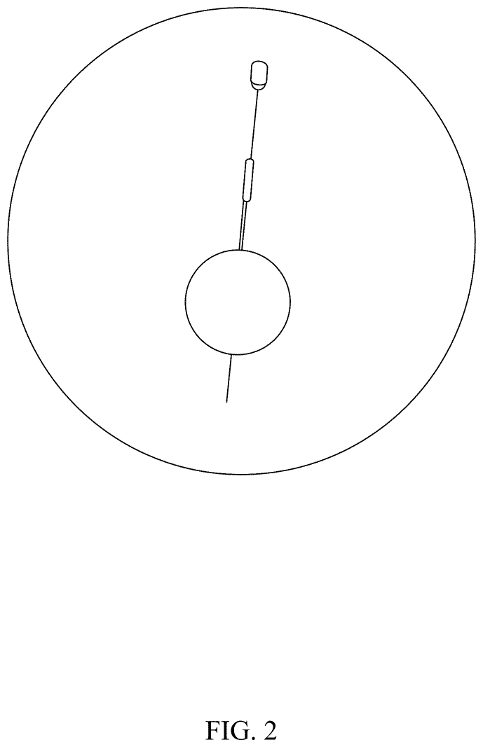

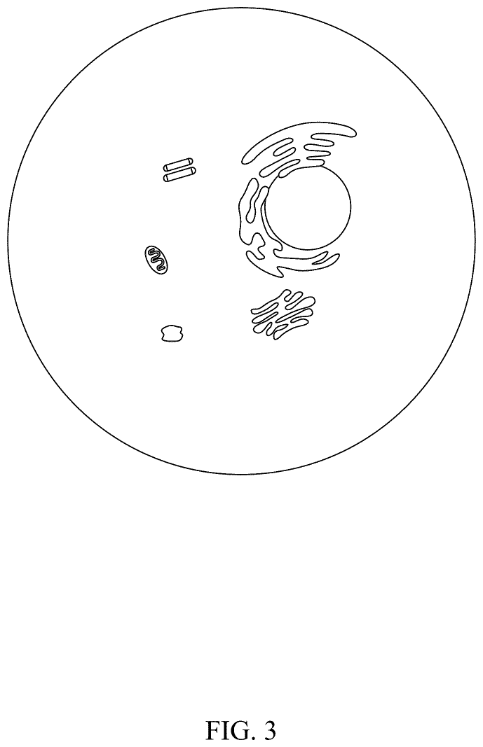

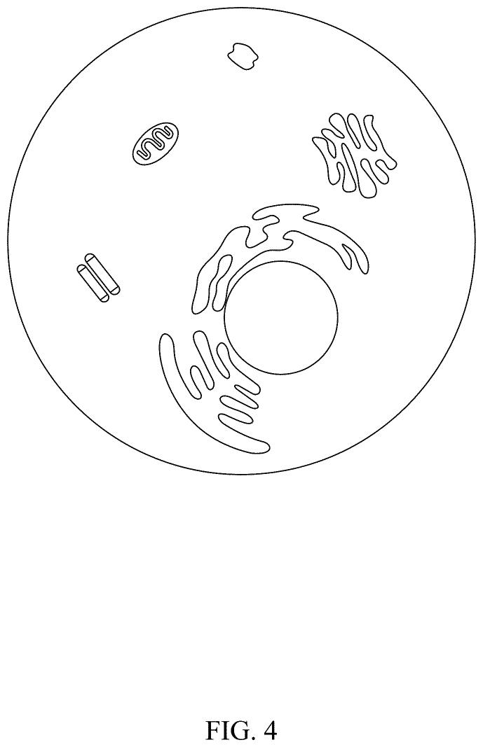

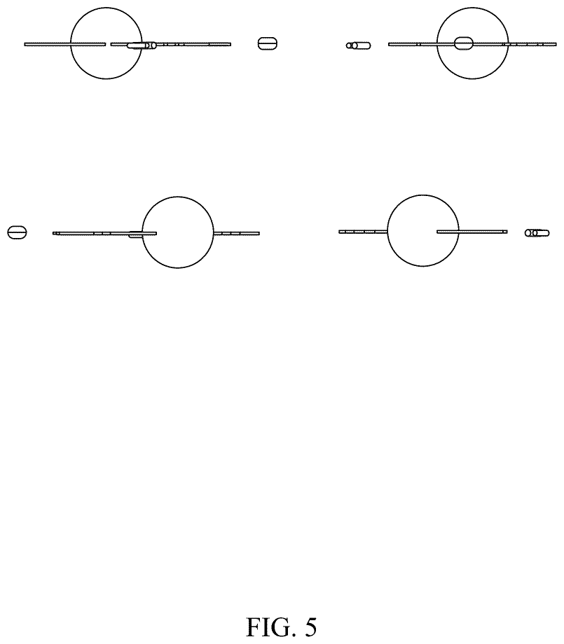

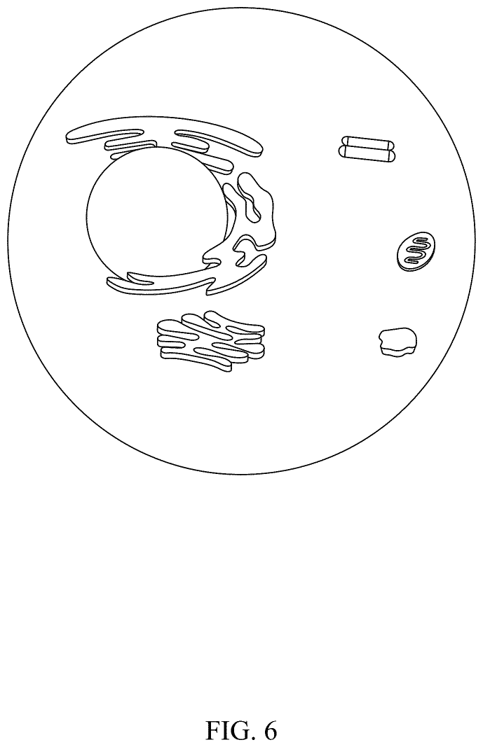

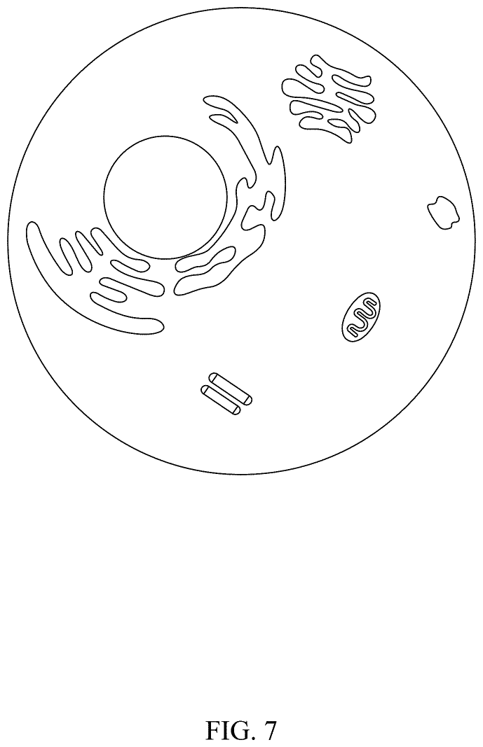

As can be seen in FIGS. 2 through 7, the present invention is directed to a fluid-encapsulated cell model that incorporates the presence of 3D-printed organelles and a mechanism to connect the macrostructural similarities to that of those in the molecular scale. The cell structure with a plurality of organelle models is configured to be manipulated during experimentation purposes and arranged on a generally flat surface, wherein the plurality of organelle models is further configured to be arranged with respect to each other so as to represent the arrangement of a plurality of organelles of an actual biological cell.

The present invention as per its preferred embodiments provides encapsulated 3D-modeled organelles. The organelles are designed to be housed in a spherical cell that allows the diffusion of small ions. The model is a transparent display of these organelles that is created in the process of spherification. The conceptualization of this model is the housing of the 3D organelles in a liquid environment to allow the experimentation of complex molecular subjects.

The present invention is a new process of visualizing the diffusion of small ions under the biological scope of cell modeling. The existence of the cell model is a new method of encapsulating 3D-printed organelles. The model is an ion-activated artificial model of biological cells. The calcium-sodium reaction generates the encapsulation process of the organelles. This process has been coined the term “spherification” and uses the example medium of sodium alginate to generate this process. After this, the sphere is generally dipped in a solution of water to wash off the outer layer and halt the process of spherification.

The present invention is a system and method of presenting a fluid-encapsulated cell model to incorporate the presence of 3D-printed organelles comprising: a first body of spherical-shaped cell; and a set of abstractly designed organelles housed within the cell body. Moreover, the spherically shaped cell body is transparent in the structure. The organelles are designed to be housed in a spherical cell and allow the diffusion of small ions. The housing of the 3D organelles in a liquid environment provides a medium for the experimentation of molecular subjects.

The present invention is also an artificial method of an ion-activated model of biological cells, wherein: the process involves the diffusion of small ions under the biological scope of cell modeling; the process involves an ion-activated artificial model of biological cells; the process involves calcium sodium reaction to generate the encapsulation process of the organelles; and the process utilizes medium of sodium alginate to generate the process.

The present invention is also a novel methodology of presenting encapsulated 3D-modeled organelles for education and technological purposes that are within a spherical-shaped cell body and are experimented within a liquid environment to study complex molecular subjects. The model is an ion-activated artificial model, wherein the calcium-sodium reaction generates the encapsulation process of organelles. The organelles are housed on a depositing device which suspends the organelles, filaments, nanobots, or particulate of interest for the model within the layering of the polymer. This process allows for the whole sphere to retain its fluid interior for the diffusion of small molecules in the process of experimentation.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

Although the invention has been explained in relation to its preferred embodiment, it is to be understood that many other possible modifications and variations can be made without departing from the spirit and scope of the invention as hereinafter claimed.

What is claimed is:

1. A fluid-encapsulated eukaryotic-cell model comprising:
a liquid environment;
a semi-permeable membrane;
a plurality of artificial organelles;
the semi-permeable membrane being immersed within the liquid environment;
an encapsulated portion of liquid from the liquid environment being located within the semi-permeable membrane;
the encapsulated portion of liquid and the plurality of artificial organelles being housed within the semi-permeable membrane; and
the plurality of artificial organelles being suspended within the semi-permeable membrane by the encapsulated portion of liquid.

2. The fluid-encapsulated eukaryotic-cell model as claimed in claim 1, wherein the semi-permeable membrane is transparent.

3. The fluid-encapsulated eukaryotic-cell model as claimed in claim 1, wherein the semi-permeable membrane is made of a polymer.

4. The fluid-encapsulated eukaryotic-cell model as claimed in claim 3, wherein the semi-permeable membrane is made of an alginate polymer.

5. The fluid-encapsulated eukaryotic-cell model as claimed in claim 1, wherein the semi-permeable membrane is a spherical shape.

6. The fluid-encapsulated eukaryotic-cell model as claimed in claim 1, wherein the semi-permeable membrane is configured to bidirectionally diffuse small molecules through a chemical mechanism used in cell encapsulation.

7. The fluid-encapsulated eukaryotic-cell model as claimed in claim 6, wherein the small molecules are small ions.

8. The fluid-encapsulated eukaryotic-cell model as claimed in claim 6, wherein the chemical mechanism used in cell encapsulation is a calcium-sodium reaction.

9. The fluid-encapsulated eukaryotic-cell model as claimed in claim 6, wherein sodium alginate is dispersed throughout the liquid environment, and wherein the sodium alginate is a medium for the chemical mechanism used in cell encapsulation.

10. The fluid-encapsulated eukaryotic-cell model as claimed in claim 1, wherein the plurality of artificial organelles is a plurality of three-dimensionally-printed organelles.

11. The fluid-encapsulated eukaryotic-cell model as claimed in claim 1 further comprising:
a depositing layer;
the depositing layer being mounted within the semi-permeable membrane; and
the plurality of artificial organelles being mounted onto the depositing layer.

12. The fluid-encapsulated eukaryotic-cell model as claimed in claim 11, wherein the depositing layer is a generally-flat shape.

13. The fluid-encapsulated eukaryotic-cell model as claimed in claim 1, wherein the plurality of artificial organelles is arranged with a specific interstitial spacing, wherein the specific interstitial spacing is proportional to an interstitial spacing amongst organelles of a naturally-occurring eukaryotic cell.

14. A fluid-encapsulated eukaryotic-cell model comprising:
a liquid environment;
a semi-permeable membrane;
a plurality of artificial organelles;
the semi-permeable membrane being immersed within the liquid environment;
an encapsulated portion of liquid from the liquid environment being located within the semi-permeable membrane;
the encapsulated portion of liquid and the plurality of artificial organelles being housed within the semi-permeable membrane;
the plurality of artificial organelles being suspended within the semi-permeable membrane by the encapsulated portion of liquid;
the semi-permeable membrane being transparent;
the semi-permeable membrane being made of a polymer;
the semi-permeable membrane being a spherical shape;
the semi-permeable membrane being configured to bidirectionally diffuse small molecules through a chemical mechanism used in cell encapsulation; and
the plurality of artificial organelles is a plurality of three-dimensionally-printed organelles.

15. The fluid-encapsulated eukaryotic-cell model as claimed in claim 14, wherein the semi-permeable membrane is made of an alginate polymer.

16. The fluid-encapsulated eukaryotic-cell model as claimed in claim 14, wherein the small molecules are small ions.

17. The fluid-encapsulated eukaryotic-cell model as claimed in claim 14, wherein the chemical mechanism used in cell encapsulation is a calcium-sodium reaction.

18. The fluid-encapsulated eukaryotic-cell model as claimed in claim 14, wherein sodium alginate is dispersed throughout the liquid environment, and wherein the sodium alginate is a medium for the chemical mechanism used in cell encapsulation.

19. The fluid-encapsulated eukaryotic-cell model as claimed in claim 14 further comprising:
a depositing layer;
the depositing layer being a generally-flat shape;
the depositing layer being mounted within the semi-permeable membrane; and
the plurality of artificial organelles being mounted onto the depositing layer.

20. The fluid-encapsulated eukaryotic-cell model as claimed in claim 1, wherein the plurality of artificial organelles is arranged with a specific interstitial spacing, wherein the specific interstitial spacing is proportional to an interstitial spacing amongst organelles of a naturally-occurring eukaryotic cell.
